(19) United States
(12) Patent Application Publication
(10) Pub. No.: US 2025/0135224 A1
(43) Pub. Date: May 1, 2025

(54) ANTIMICROBIAL LIGHT-EMITTING DEVICE AND METHOD FOR CATHETERS

(71) Applicant: Luminary, LLC, Marlborough, MA (US)

(72) Inventors: Adam E. M. Eltorai, Marlborough, MA (US); Charles W. Henry, Denver, CO (US)

(21) Appl. No.: 18/944,280

(22) Filed: Nov. 12, 2024

Related U.S. Application Data
(63) Continuation-in-part of application No. 17/851,562, filed on Jun. 28, 2022.
Application No. 17/851,562 is a continuation-in-part of application No. 17/665,965, filed on Feb. 7, 2022, now Pat. No. 11,596,772.
(60) Provisional application No. 63/548,080, filed on Nov. 10, 2023.
(60) Provisional application No. 63/146,697, filed on Feb. 7, 2021.
(60) Provisional application No. 63/215,910, filed on Jun. 28, 2021.

(51) Int. Cl. A61N 5/06

(52) U.S. Cl. CPC A61N 5/0624; A61N 2005/0632; A61N 2005/0645; A61N 2005/0651

(57) ABSTRACT

An antimicrobial-light emitting light device comprising a skin-safe antimicrobial light source, power source, and an attachment for use with catheter dressings or catheter securement devices. A method of reducing catheter-related infections, comprising providing a skin-safe antimicrobial light-emitting device, positioning the antimicrobial light to bathe the skin entry site of a catheter, and reducing the bacterial burden in the region around the skin entry site of a catheter.

### RELATED APPLICATIONS

This Patent Application claims the benefit under 35 U.S.C. § 119 (e) of U.S. Provisional Patent App. No. 63/548,080 filed Nov. 10, 2023, entitled “ANTIMICROBIAL LIGHT-EMITTING DEVICE, SYSTEM, AND METHOD FOR PERIPHERAL INTRAVENOUS CATHETERS”, and is a Continuation-in-Part (CIP) of U.S. patent application Ser. No. 17/851,562, filed Jun. 28, 2022, entitled “ANTIMICROBIAL LIGHT-EMITTING OROPHARYNGEAL DEVICE FOR VENTILATOR-ASSOCIATED PNEUMONIA,” which is a Continuation-in-Part (CIP) of U.S. patent application Ser. No. 17/665,965, filed Feb. 7, 2022, entitled “ANTIMICROBIAL LIGHT-EMITTING PERCUTANEOUS SITE DRESSING,” which claims the benefit under 35 U.S.C. § 119 (e) of U.S. Provisional Patent App. No. 63/146,697, filed Feb. 7, 2021, entitled “ANTIMICROBIAL LIGHT DRESSING DEVICE,” and U.S. Provisional Patent App. No. 63/215,910, filed Jun. 28, 2021, entitled “ANTIMICROBIAL LIGHT-EMITTING DEVICES,” all incorporated herein by reference in entirety.

### BACKGROUND

Catheter-related infections are a significant concern in medical environments, as they can lead to severe complications, including bloodstream infections, prolonged hospital stays, and increased healthcare costs. Central venous catheters (CVCs), peripheral intravenous catheters (PIVCs), and other invasive lines create a direct path for bacteria to enter the body, and the dressing and securement site around these lines are particularly vulnerable to bacterial colonization. Despite improvements in catheter materials, insertion techniques, and dressing protocols, infections remain a critical issue.

Current methods for reducing catheter-related infections primarily involve the use of sterile dressings, antimicrobial solutions, and routine dressing changes. However, these strategies have limitations, particularly when it comes to maintaining continuous, effective antimicrobial action without disrupting the site. Emerging technologies, such as antimicrobial light-emitting devices, show promise in providing an additional layer of infection control by continuously targeting and reducing bacterial colonization at the catheter insertion site. Light, specifically in wavelengths with antimicrobial properties, has been demonstrated to effectively inhibit the growth of common pathogens without the need for physical contact or chemical application, minimizing disruption to the dressing site and potentially enhancing patient comfort.

### SUMMARY

The invention provides an antimicrobial light-emitting device designed for catheter dressings and catheter securement devices. This device integrates a light source that emits light at wavelengths effective against a range of bacteria, fungi, and other pathogens commonly associated with catheter-related infections. The light source is attached to or embedded within the dressing or securement device, allowing for continuous or periodic light exposure to the catheter insertion site without requiring removal of the dressing.

The light is preferably in wavelengths known for both antimicrobial effects while being safe to humans, such as antimicrobial blue light or far UV-C light, and is delivered at safe, non-toxic levels for human tissue. This design provides a targeted, non-invasive method for reducing microbial load at the catheter site, potentially decreasing the incidence of catheter-related infections. The device is compatible with various catheter types, dressings, catheter securement devices, and can be easily integrated into standard catheter care protocols, offering a practical and effective solution for enhancing patient safety.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context view of a medical treatment environment suitable for use with configurations herein;

FIG. 2 is a perspective view of the medical device for antimicrobial light treatment of a percutaneous insertion site;

FIGS. 3A-3C show engagement of the device of FIG. 2 with a treatment region defined by the percutaneous insertion site;

FIGS. 4A-4B show perspective views of a central void in the device of FIGS. 1-3c;

FIG. 5 shows a plan view of the device of FIGS. 1-4B;

FIG. 6 shows a side, cutaway view of the central void and illumination cavity formed by the device of FIGS. 1-5;

FIG. 7 shows a perspective view of the illumination cavity of FIG. 6;

FIG. 8 shows a bottom view of the device of FIGS. 1-7;

FIG. 9 shows an underside perspective view of the device and illumination/light cavity of FIGS. 6-8;

FIG. 10 shows a method of applying the antimicrobial light treatment of FIGS. 1-9;

FIG. 11 shows an example context of a catheter, central line or IV insertion site suitable for use with configurations herein;

FIG. 12 shows an alternate configuration for antimicrobial light treatment of a percutaneous insertion site as in FIG. 2;

FIG. 13 shows a freestanding device for antimicrobial light treatment as in FIG. 12;

FIG. 14 shows an epidermal mounting service on an adhesive tape substrate;

FIG. 15 shows the antimicrobial light-emitting device of FIG. 13 in a more compact housing;

FIG. 16 shows the configuration of FIG. 12 in a deployment scenario;

FIGS. 17A-17F show an alternate compact configuration of the device of FIG. 15;

FIGS. 18A-18C show alternate configurations of the device of FIG. 12;

FIGS. 19A-19B show alternate configurations based on the embodiment of FIG. 15;

FIG. 20 shows available antibacterial light wavelengths that the LED may render;

FIG. 21 shows the adhesive strip in further detail; and

FIGS. 22A-22C show use cases of deployment on the patient skin surface.

### DETAILED DESCRIPTION

A device for the dressing of wounds and insertion sites of percutaneous and drug delivery devices provides circumferential protection of a wound or insertion site of a percutaneous or drug delivery device. In particular, the device is an integrated dressing for vascular and non-vascular percutaneous medical devices (e.g., IV catheters, central venous lines, arterial catheters, dialysis catheters, peripherally inserted coronary catheters, mid-line catheter, drains, chest tubes, externally placed orthopedic pins, ventricular assist device drivelines, and epidural catheters) comprising an adhesive dressing and an antimicrobial light source, such as visible light, far UVC light, and any suitable electromagnetic emission of a therapeutically beneficial wavelength. The dressing device reduces infection risk from vascular and non-vascular percutaneous medical devices by providing sufficient tissue-safe antimicrobial light at a wound or insertion site.

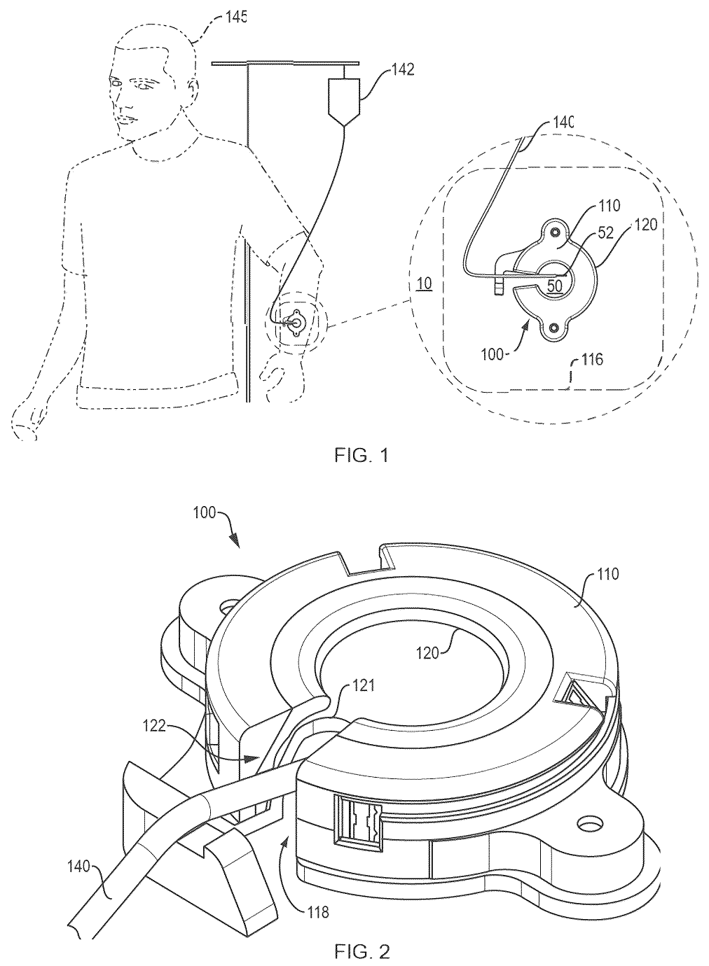

FIG. 1 is a context view of a medical treatment environment suitable for use with configurations herein. Referring to FIG. 1, an antimicrobial epidermal device 100 includes a circumferential light-emitting body 110 configured for adhesion around a percutaneous insertion site 52 for directing therapeutic light at the percutaneous insertion site while permitting unobstructed passage of a medication vessel 140 to the percutaneous insertion site 52. The medication vessel emanates from a fluidic source 142 of medication or other liquid, such as an IV (Intravenous) bag. The percutaneous insertion site 52 defines a surrounding treatment region 50, typically on an arm of a patient 145 because of ease of IV access, however any suitable epidermal region may be selected for the percutaneous insertion site.

In the antimicrobial epidermal device 100, the circumferential body 110 is adapted for epidermal placement on the treatment region 50 of a larger epidermal surface 10. Placement is based on a central void 120 in the circumferential body for epidermal access and alignment generally over the insertion site 52. The circumferential body 110 includes an illumination source disposed for emitting a therapeutic light on the treatment region 50 defined by the central void 120. An adhesive member 116, such as a patch or bandage, adheres the circumferential body 110, vessel 140 and a percutaneous penetration member such as a needle to the epidermal area around the treatment region 50.

FIG. 2 is a perspective view of the medical device of FIG. 1 for antimicrobial light treatment around a percutaneous insertion site 52. FIG. 2 shows the central void 120 accessible by a vessel gap 122 in the circumferential body 110 for passage of the medication vessel 140 to a penetration or insertion member defining the insertion site 52. The treatment region 50 is defined by a radius around the insertion site roughly centered within the circumferential body. In the example of FIG. 2, the central void 120 remains covered by an insert 121 except at the vessel gap 122 for permitting vessel access into an illumination cavity 118.

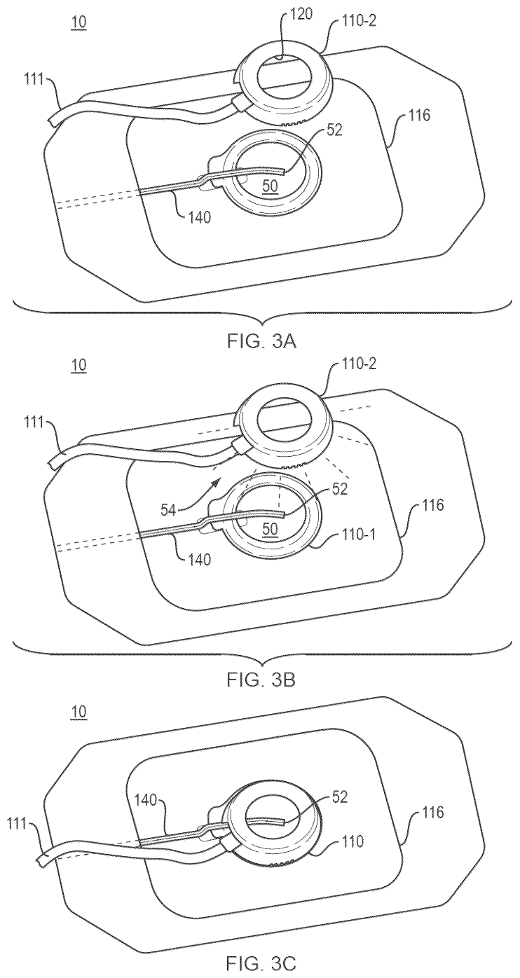

FIGS. 3A-3C show engagement of the device of FIG. 2 with a treatment region defined by the percutaneous insertion site. In a particular configuration, the device may be combined with an adhesive member 116 such as a sheet, patch or bandage for providing a system of secure attachment of the illumination source to the percutaneous insertion site. Referring to FIGS. 1-3C, the circumferential body 110 is disposed on an epidermal surface 10, in conjunction with an adhesive member 116. The adhesive member 116 has an adhesive attraction to the epidermal surface 10 and extends over the treatment region 50 and is disposed for securing the circumferential body 110 and a treatment vessel 140 directed to the central void 120. The adhesive member my include a securement or fixation dressing having adhesive and therapeutic or antimicrobial properties. The securement or fixation dressing is disposed between the circumferential body 110 and the epidermal surface 10. The circumferential body is therefore disposed in place by the underlying securement or fixation dressing/patch, and substantially centered around the insertion site.

The configuration of FIG. 3 shows a two-part configuration of the device. The circumferential body 110 further includes a distal layer 110-2 including a power connection 111 for powering an illumination source such as one or more LED elements and a proximate layer 110-1 having a translucent surface, in which the LED elements are disposed within the distal layer 110-2 for directing the therapeutic light onto the treatment region 50.

The proximate layer 110-1 engages with the adhesive member 116, which may be integrated as an adhesive whole or applied in separate phases. In the configuration of FIGS. 3A-3C, the adhesive member 116 may reside between the proximate layer 110-1 and distal layer 110-2. The adhesive member 116 secures the insertion member at the insertion site 52 alone with the medication vessel 140, shown in FIG. 3A. The treatment region 50 is defined by a radius around the dermal insertion site 52, where the insertion site 52 provides the dermal access for medical intervention through the skin by a sharp, piercing structure.

In FIG. 3B, the distal layer 110-2 approaches the secured, proximate layer 110-1. The distal layer 110-2 may already be emitting light 54 onto the treatment region 50. In FIG. 3C, the distal layer 110-2 engages the proximate portion 110-2 to form the full circumferential member 110, and encapsulates an illumination cavity, discussed further below.

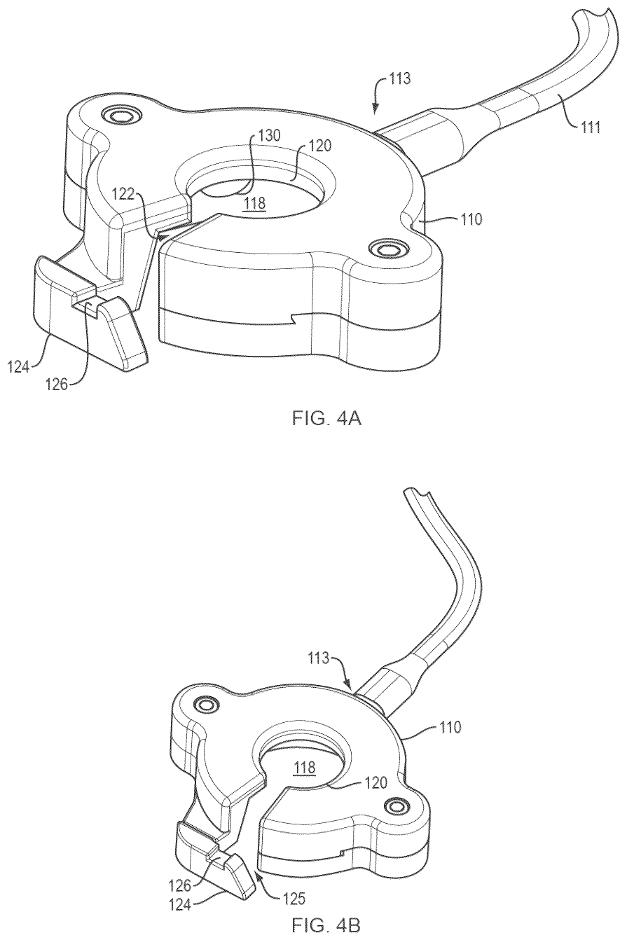

FIGS. 4A-4B show perspective views of a central void 120 in the device of FIGS. 1-3c. Referring to FIGS. 1-4B, upon adherence and proper administration, the circumferential body 110 adheres to the epidermal surface 10 with the central void 120 roughly centered on the insertion site 52. An illumination source 130 includes at least one LED element defining the illumination source, in which the LED element emits a wavelength based on an antimicrobial effect. The central void 120 has a size based on a treatment vessel 140 size and clearance over the insertion site 152. The treatment vessel 140 has an attachment to the insertion member such as a needle for a percutaneous insertion under the central void. The vessel extends through the vessel gap 122 and through the central void 120 or at least through the gap 122 and into the illumination cavity 118.

A power connection 113 receives the power supply 111 on the circumferential body 110. The power supply couples to the illumination source 130 and is adapted for receiving an electrical source for powering the illumination source, such as an external USB, batteries, AC or similar AC or DC source based on the electrical requirements of the illumination source 30. A discontinuity in the circumferential body defines the vessel gap 122 for accommodating the treatment vessel 140. The treatment vessel 140 couples to the percutaneous insertion member in the treatment region 50 under the central void 120. Routing of the treatment vessel 140 is provided by a protrusion 124 extending outward from the circumferential body. The protrusion 124 has an elevated surface 126 disposed away from the epidermal surface 10, such that the elevated surface 126 is adjacent the vessel gap 122 for directing the treatment vessel at an offset distance from the dermal surface 10. Elevation of the treatment vessel 140 above the skin avoids a shadow from the light and instead allows a shadowed region 125 to be reached by light from the illumination source 130 rather than being shaded or obscured by the vessel 140 from reaching the skin at the shadowed region.

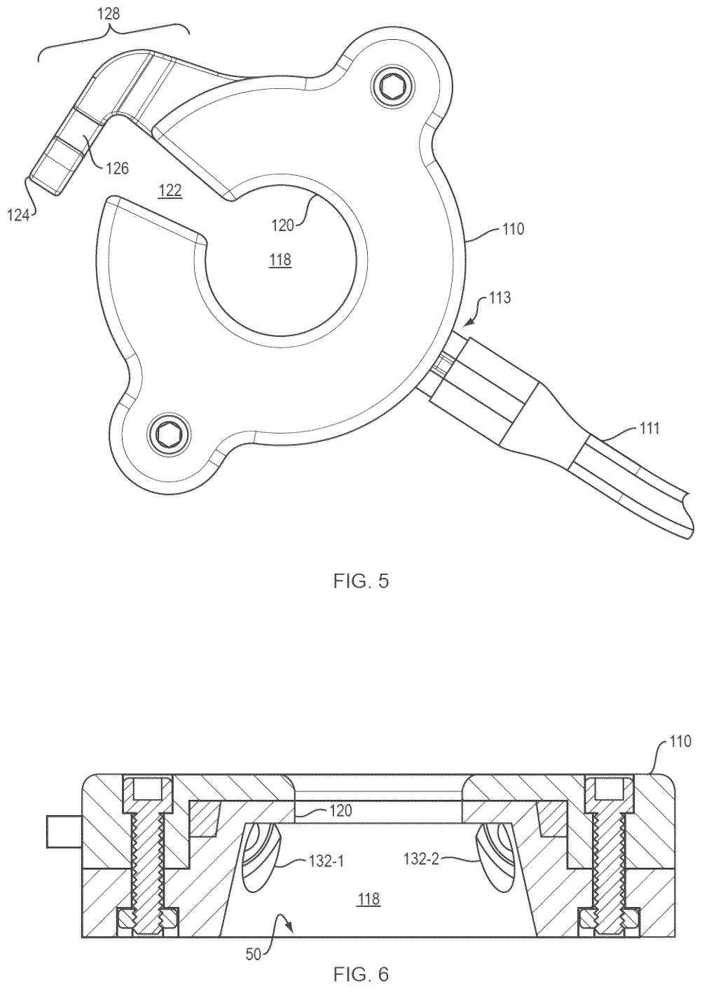

FIG. 5 shows a plan view of the device of FIGS. 1-4B. Referring to FIGS. 1-5, the vessel gap 122 is an opening or passage in the circumferential body 110. A lateral extension 128 extends radially from the circumferential body 110 adjacent the vessel gap 122, and turns toward the gap 122 to provide the elevated surface 126 residing on the protrusion 124. The elevated surface 126 is disposed on a path towards the central void 120 for receiving a treatment vessel 140 disposed on the path for fluidic delivery to an insertion site 52 in the treatment region 50.

FIG. 6 shows a side, cutaway view of the central void and light cavity formed by the device of FIGS. 1-5. Referring to FIGS. 1-6, a plurality of LED elements 132-1 . . . 132-2 (132 generally) surround the illumination cavity 118, although as few as I could be provided. In the example configuration, the plurality of LED elements 132 are disposed generally in a circle around the circumferential body 110, and fill the illumination cavity 118 with light focused on the treatment region 50. The inner surface of the circumferential body 110 and optional insert 121 are a light color and may be translucent to reflect and refract (distribute and target) as much if the light as possible around the illumination cavity 118 to fall on the treatment region 50. The antimicrobial light is therefore specifically targeted to fall on the treatment region defined by the percutaneous insertion and surrounding epidermal region, specifically within the illumination cavity 118 of the circumferential body 110.

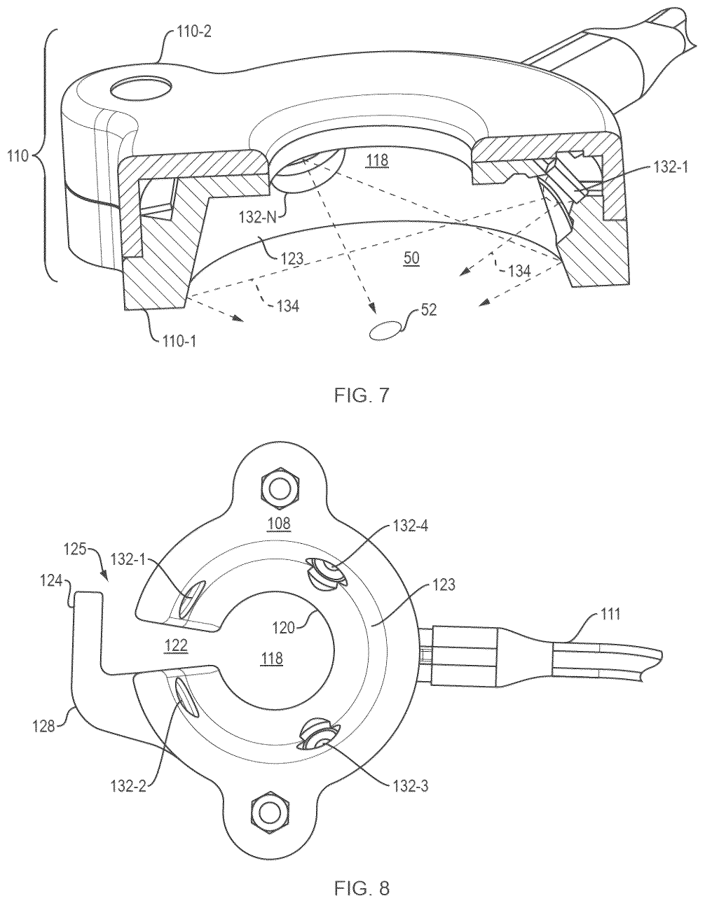

FIG. 7 shows a perspective view of the light cavity of FIG. 6 as a cutaway from the circumferential body 110. Referring to FIGS. 1-7, the circumferential body 110 is disposed on a treatment region 50 and centered on or around an insertion site 52 of a percutaneous insertion member. One or more LED elements 132-N disposed on an inner surface 123 of the circumferential body bathe the illumination cavity 118 in light for directing the light directly on the treatment region 50 and also reflected and/or refracted around the inner surface 123 as shown by arrows 134. A light colored, translucent and/or reflective property of the inner surface 123 generally focuses direct and indirect light onto the treatment region 50 for eliminating harmful pathogens that may live on the skin surface around the insertion site 52.

FIG. 8 shows a bottom view of the device of FIGS. 1-7. Referring to FIGS. 7-8, FIG. 8 shows four LEDs 132-1 . . . 132-4 emanating from the inner surface 123, however any suitable number of LEDs may be provided based on the intensity and wavelength of the therapeutic light sought for irradiation. Any suitable propagated wavelength of the electromagnetic spectrum may be provided if an illumination element can be so equipped. The underside 108 rests on the dermal surface 10 at the treatment region, adhered by the adhesive member 116 as discussed above. The protrusion 124 has a bottom flush with the underside 128, and opens to define the illumination cavity 118. The lateral extension 128 is flush with the underside 128 for resting on the skin surface, and extends in an articulated manner for protrusion 124 to form the elevated surface 126 at the vessel gap 122.

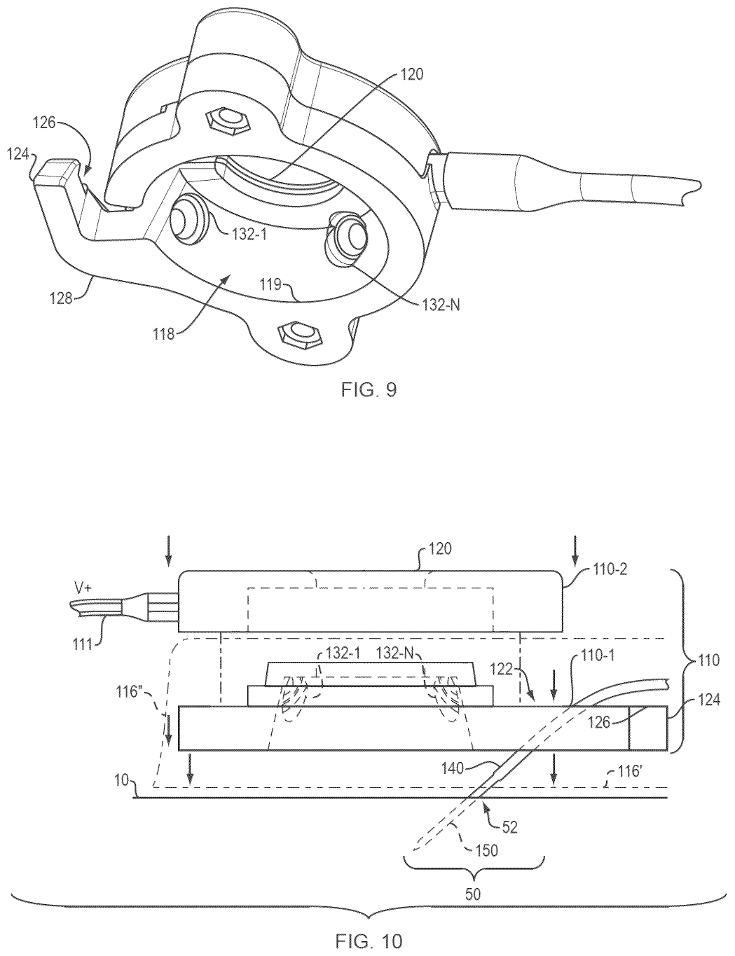

FIG. 9 shows an underside perspective view of the device and illumination cavity 118 of FIGS. 6-8. The illumination cavity 118 is based on a generally concave region under the central gap 120 and extending to an inner perimeter 119 of the circumferential body 110, with the vessel gap 122 allowing passage of the treatment vessel 140.

FIG. 10 shows a method of applying the antimicrobial light treatment of FIGS. 1-9. Referring to FIGS. 1-10, a method for antimicrobial light treatment of a percutaneous insertion site as shown in FIG. 10 includes applying an adhesive member 116 to a treatment region 50 for securing a percutaneous insertion member in an insertion site. The percutaneous insertion member 150, such as a needle, is in fluidic communication with a medication vessel 140 for delivering medication, typically an IV line, infusion line or similar delivery system. The adhesive member 116 may adhere on the epidermal surface, shown as dotted line 116′, or may be applied over the circumferential member 110, shown as dotted line 116″. In the alternate configuration of FIGS. 3A-3B, the adhesive member 116″ may reside between the proximate layer 110-1 and distal layer 110-2.

In either configuration, the circumferential body 110 is disposed onto the treatment region 52. The circumferential body 110 extends generally circular around a central void 120, and placement centers the central void around the insertion site so that the central void allows clearance for the medication vessel 140 and any uninserted portion of the rigid insertion member. The circumferential body 110 may be any suitable shape and size based on the treatment region 50 and the intensity of the illumination source 130 thereby irradiating the treatment region.

The circumferential body 110 includes a discontinuous portion defining the vessel gap 122, which may be continuous with the central void 120. In conjunction with placement of the circumferential body 110, the medication vessel is routed over the elevated surface 126 on the protrusion 124 extending from the circumferential body for permitting the vessel to extending through the vessel gap 122 above and out of contact with the skin surface. The treatment region 50 is illuminated from one or more LEDs (Light Emitting Diodes) 132 disposed on an inner surface of the circumferential body 110 for irradiating an illumination cavity 118 defined by the inner surface and the central void. The LEDs 132 or other illumination source irradiate the treatment region for maintaining an antimicrobial and sterile environment around the insertion site 50. This prevents pathogens from entering the patient along the insertion member 150.

In a particular configuration shown in FIGS. 3A-3C above, the circumferential body has multiple, engageable parts. A first, proximate layer 110-1 accompanies the insertion member 150. Disposing the circumferential body 110 further comprises disposing the proximate layer 110-1 by applying a proximate layer centered on the treatment region using the adhesive member 116, and engaging the distal layer 110-2 onto the proximate layer 116-1 by circumferentially aligning the distal layer with the proximate layer, the LEDs directed towards the illumination cavity. Any suitable adhesive, friction, interference and/or deformable (i.e. snap-fit plastic tab) mechanism may be employed for engaging the proximate 110-1 and distal 110-2 layers.

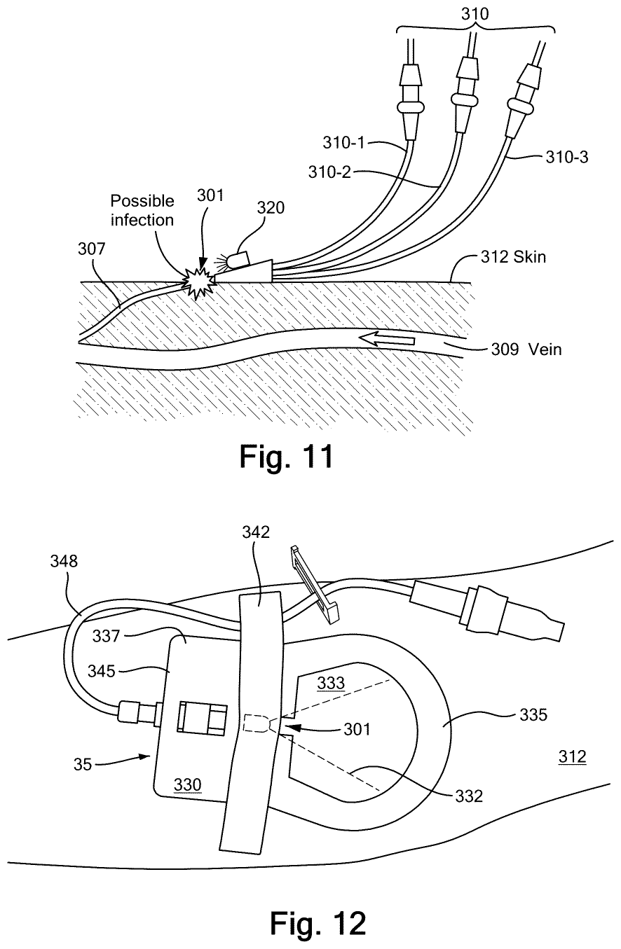

FIG. 11 shows another example context of a catheter, central line or IV insertion site suitable for use with configurations herein. Referring to FIG. 11, an insertion site 301 receives one or more percutaneous delivery vessels 310-1 . . . 310-3 (310 generally), usually terminating in a catheter or needle for piercing a patient skin surface 312 at the percutaneous insertion site 301. Typically the inserted catheter or needle fluidically engages with a blood vessel 309 or other vascular structure. An illumination source 320 persistently illuminating the insertion site 301 eradicates pathogens and bacteria through an antibacterial property of the light emanating from the illumination source 320, such as a blue light or UV (ultraviolet) light source as described above. Other suitable light sources may be selected based on antibacterial efficacy, such as far UVC light in the range of 200 nm to 230 nm, and more particularly 222 nm.

FIG. 12 shows an alternate configuration for antimicrobial light treatment of a percutaneous insertion site 301 as in FIG. 2. The percutaneous delivery vessel 310 is often taped or adhered to a skin surface 312 such as a wrist or arm. Some sites may be unwieldy for the apparatus of FIGS. 1-10, and are more suited to a low profile, taped or adhered approach. Referring to FIG. 12, an antimicrobial light-emitting device 325 includes an epidermal mounting surface 330 configured for adherence at the percutaneous insertion site 301. The illumination source 320 is disposed on the epidermal mounting surface 330 and directed at the percutaneous insertion site 301. The epidermal mounting surface 330 is part of an adherable dressing 345 for engagement with the patient skin surface 312. The adherable dressing 345 may employ an adhesive underside for adherence to the patient skin surface, and/or may be further supplemented by an adhesive strip 342 such as tape.

In a particular configuration, the epidermal mounting surface 330 is configured to receive the percutaneous delivery vessel 310, and to secure the percutaneous delivery vessel 310 within a luminary range 332 of the illumination source 320. The small, nonintrusive illumination source 320 resides on the epidermal mounting surface 330 in a position such that the mounting surface 330 does not block or obscure a luminary range 332 to the insertion site 301. For example, the illumination source 320 may be defined by an LED positioned at an edge of the adhesive strip 342 adjacent the insertion site.

In the configuration of FIG. 12, the adherable dressing 345 forms an outer radial portion 335 surrounding an inner adhering portion 333, in which the inner adhering portion 333 is formed from an adhesive, transparent film configured for adhesion at the percutaneous insertion site 301, often covering and adhering/securing the percutaneous delivery vessel 310. The transparent portion 333 ensures that the epidermal mounting surface 330 does not impede the luminary range 332 and allows the illumination source 320 to irradiate the insertion site 301. In this manner, the outer radial portion 335 forms a circumferential loop, and further defines a receptacle 340 in the outer radial portion 335. The receptacle 340 forms a small cutaway or void configured to engage or flank the percutaneous delivery vessel 310 while allowing light to pass to the vessel 310 and insertion site 301. Typically, the percutaneous delivery vessel 310 is formed from an elongated tube 348 that terminates in the rigid needle 307. The rigid needle 307 or catheter is adapted for percutaneous incision and forms a continuous enclosed fluid volume with the elongated tube 348, as is common with IV (intravenous) administered medications.

The outer radial portion 335 may merge with a tab 337, such that the tab 337 extends from the outer radial portion 335 and is configured for engaging the percutaneous delivery vessel 310 for support and/or additional adherence. In the event that the tab 337 adheres or covers the vessel 310, the tab has a void or receptacle 340 for exposing a portion of the percutaneous delivery vessel 310 when the percutaneous delivery vessel is disposed between the tab 337 and the patient skin surface 312. The illumination source 320 may also be disposed on the tab 337 and directed at the receptacle 340 for illuminating the percutaneous insertion site 301.

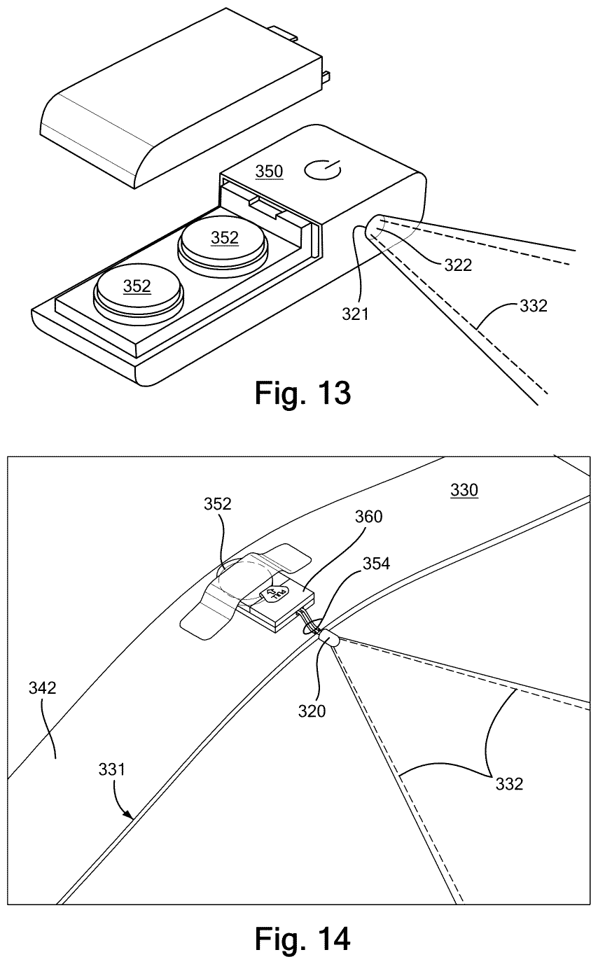

FIG. 13 shows a freestanding device for antimicrobial light treatment as in FIG. 12. Referring to FIGS. 12 and 13, the illumination source 320 further comprises a housing 350 disposed on the epidermal mounting surface 330. A power supply 352 such as one or more batteries is disposed in the housing 350 and connected for powering an LED 322 (light emitting diode) disposed in the housing 350 for defining the illumination source 320. An aperture 321 in the housing 350 directs the illumination source to the percutaneous insertion site 301

FIG. 14 shows an epidermal mounting service on an adhesive tape substrate. In some configurations, the adhesive strip 342 nay be the sole adherence mechanism to the patient skin surface 312. In such a configuration, a battery disposed on the epidermal mounting surface defines the power supply 352, and a plurality of conductors 354 extend to the illumination source 320. The illumination source 320 is disposed at an edge 331 of the epidermal mounting surface 330, in which the plurality of conductors 354 extends to and secures the illumination source 320 at the edge 321 for casting the luminary range 332 on the insertion site 301.

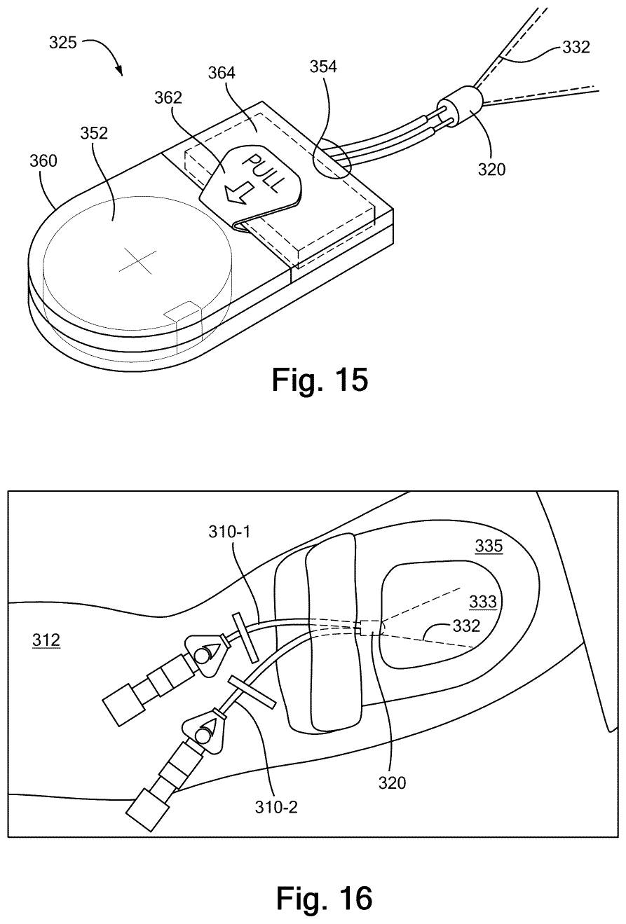

FIG. 15 shows the antimicrobial light-emitting device 325 in a more compact housing 360 including an activation tab 362 for energizing a control circuit 364 by closing a circuit with the battery, The pair of conductors 354 extends to the illumination source 320, typically having a length to orient the illumination source 320 at the edge 331.

FIG. 16 shows the configuration of FIG. 12 in a deployment scenario. Multiple delivery vessels 310-1 . . . 310-2 engage the adherable dressing 345 adjacent the illumination source 320, where the luminary range 332 casts antimicrobial light through the transparent portion 333.

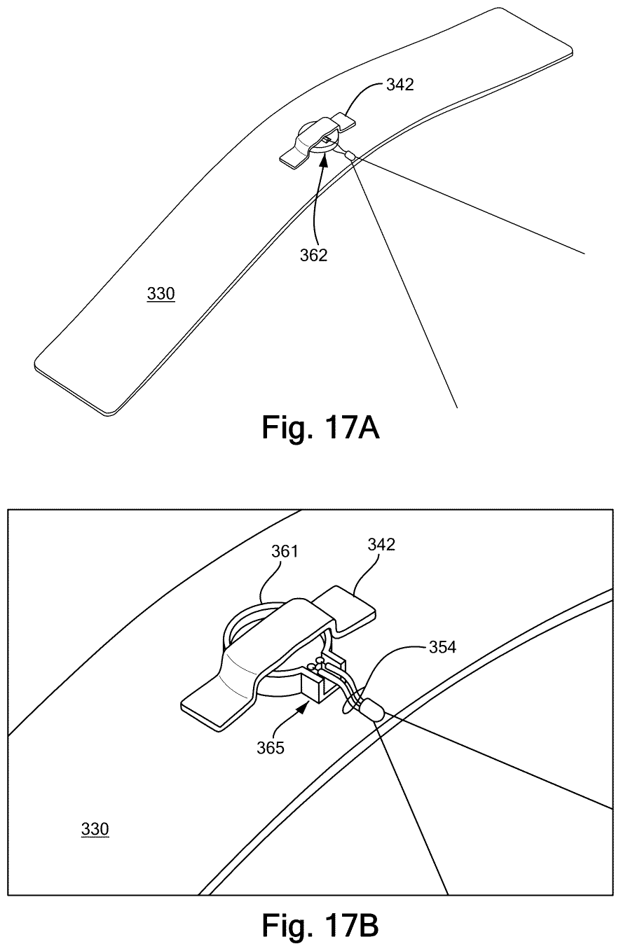

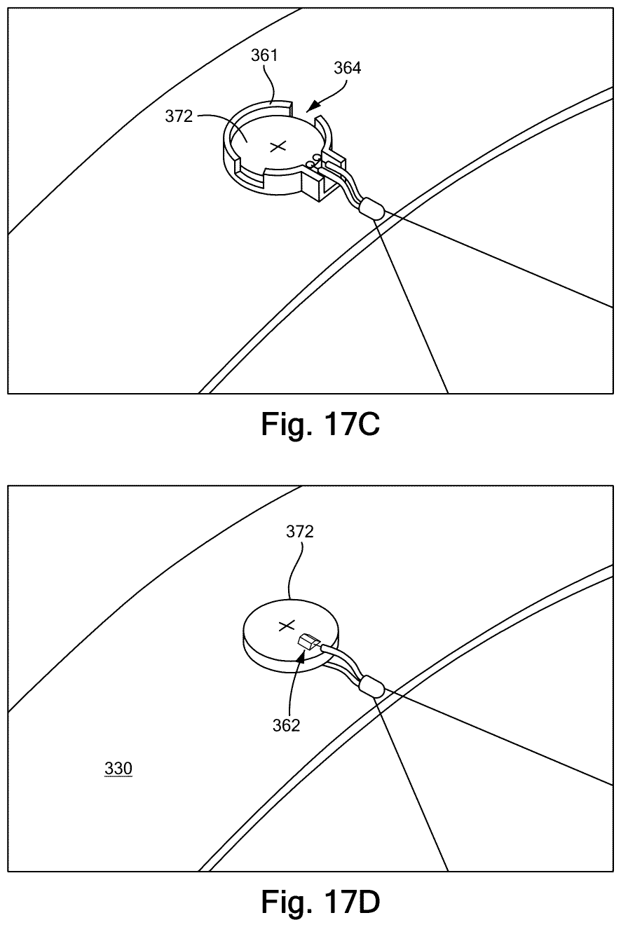

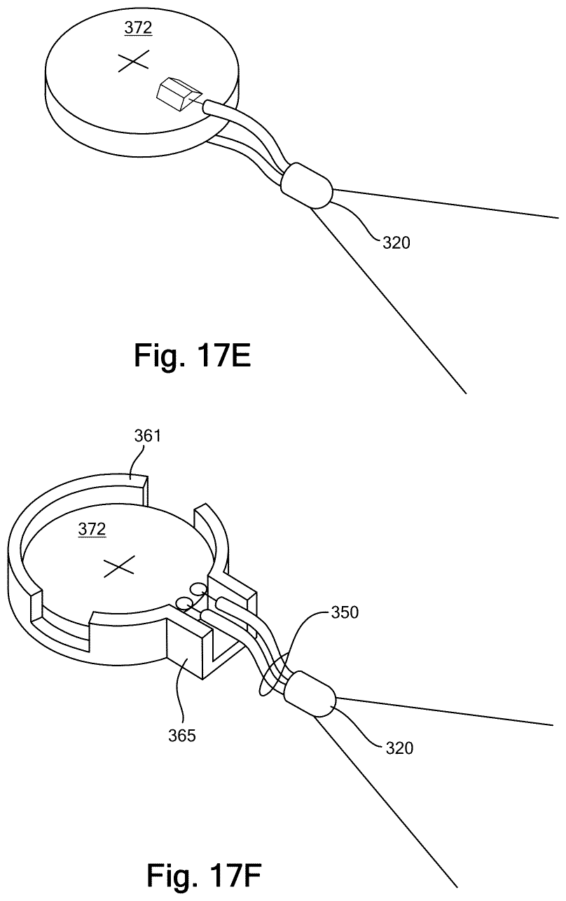

FIGS. 17A-17F show an alternate compact configuration of the device of FIG. 15. Referring to FIGS. 15 and 17A-17D, the housing 360 may be reduced or eliminated to a battery cage 361 or battery contacts 362. Recesses 364 in the battery cage 361 simplify battery insertion. It is expected that the power supply 352 is amply provided by a battery 372, such as a 3 volt coin cell common in smaller electronics. Alternately, the battery 372 may be directly adhered to the adhesive mounting surface 330. A contact enclosure 365 may also be employed to facilitate the engagement or connection to the conductors 354.

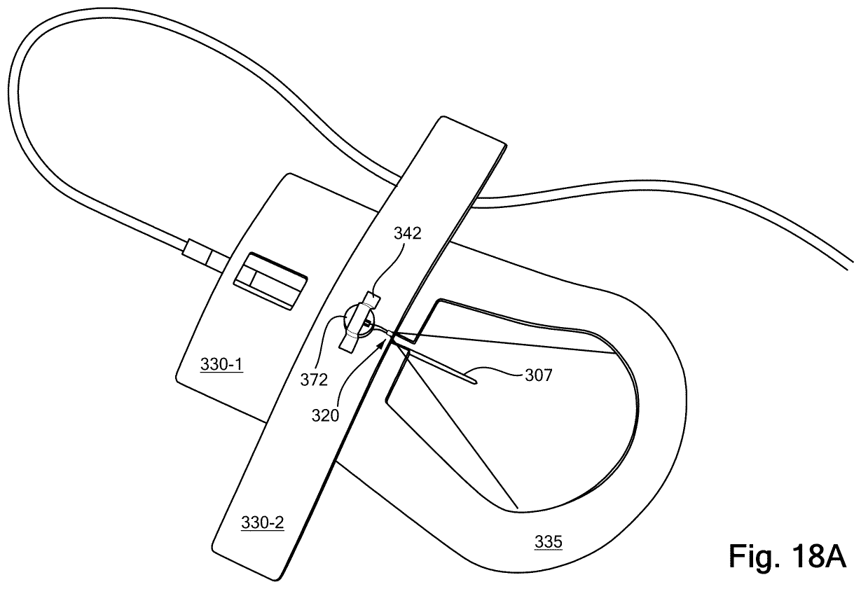

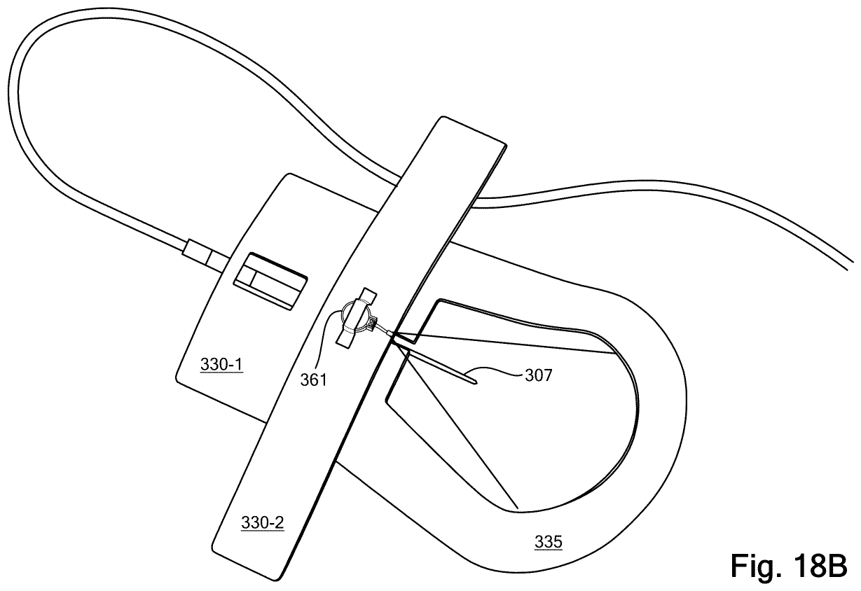

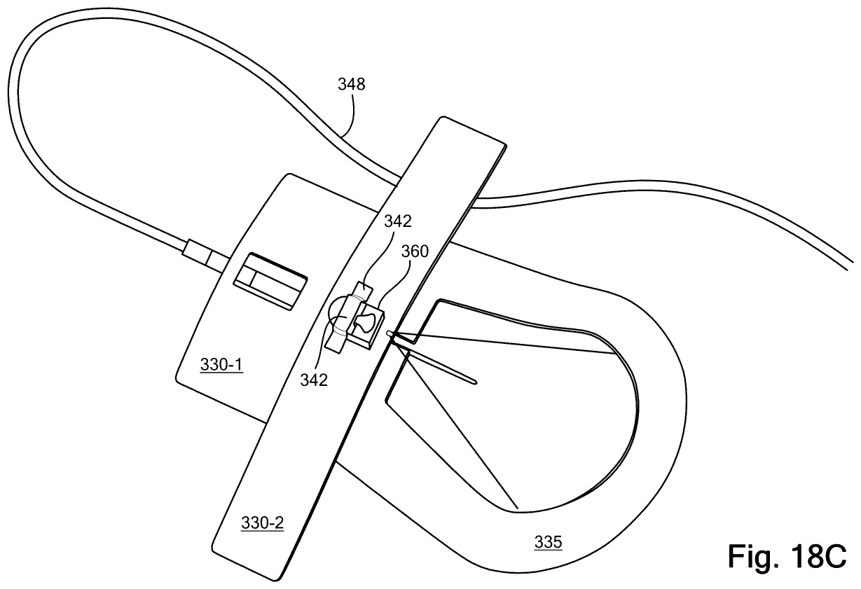

FIGS. 18A-18C show alternate configurations of the device of FIG. 12. Referring to FIGS. 12 and 18A-18C, it is apparent that the epidermal mounting surface 330 may take a variety of forms, such as an elongated strap or a circumferential ring, and may be combined for greater support for the tethered medication vessels or tubes 348. FIGS. 18A-18B depict multiple epidermal mounting surfaces 330-1 . . . 330-2 (330 generally) for locating the illumination source 320 LED adjacent alongside or on top of the needle 307 or catheter.

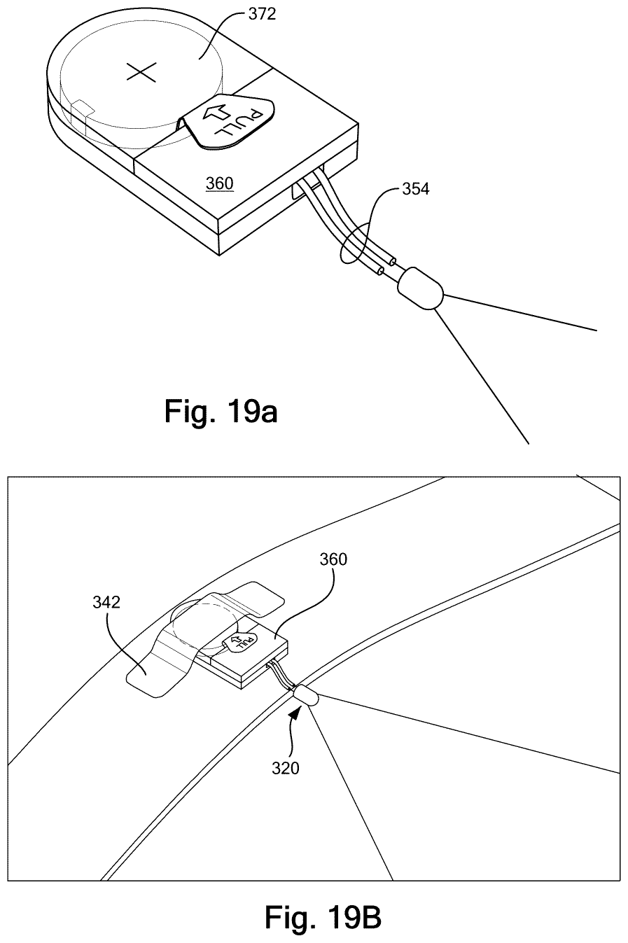

FIGS. 19A-19B show alternate configurations based on the embodiment of FIG. 15 including the housing 360.

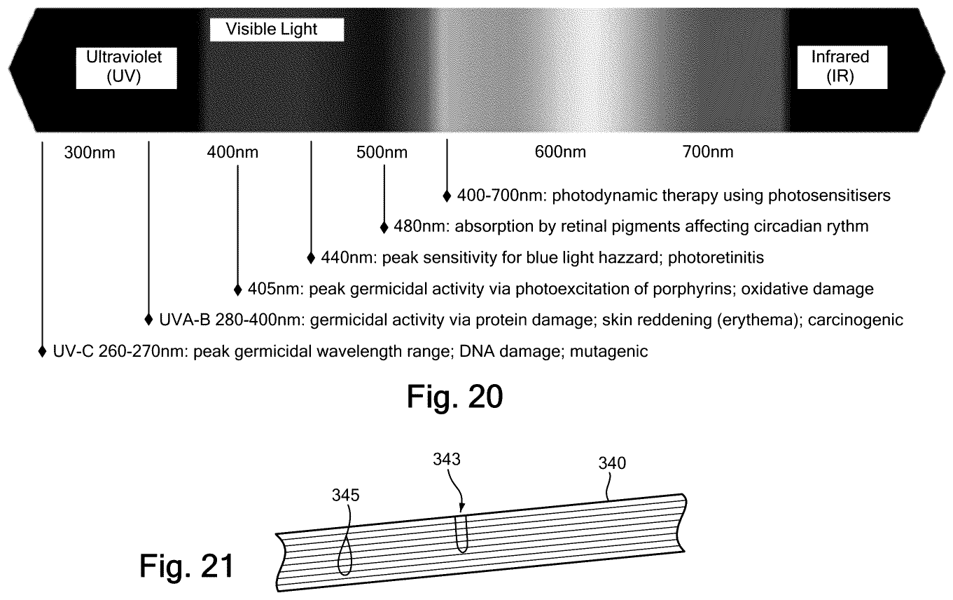

FIG. 20 shows available antibacterial light wavelengths that the LED may render;

FIG. 21 shows the adhesive strip 342 in further detail. The adhesive strip may be manually applied and may have strengthening fibers 345 for longevity and holding power. An LED segment 343 may be amenable to locating the illumination source 320.

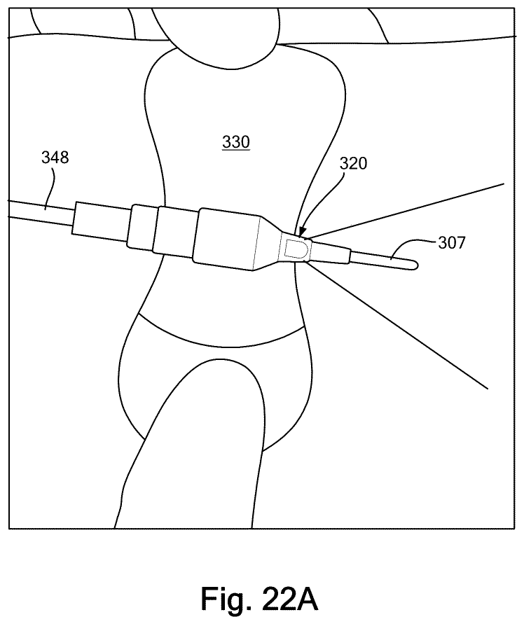

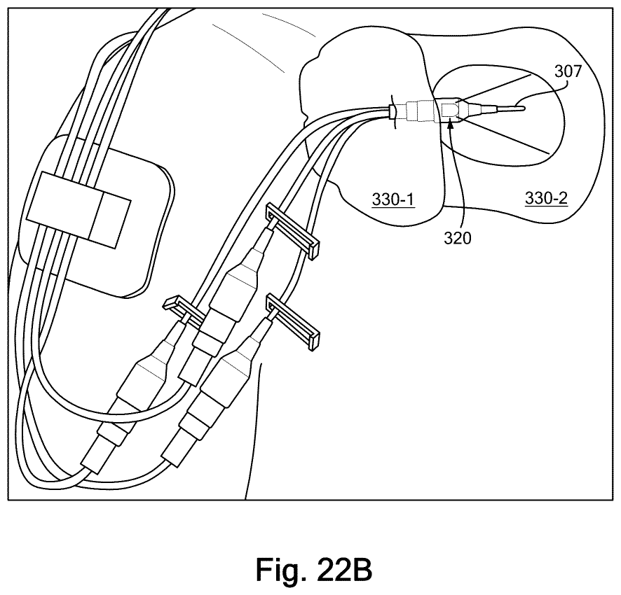

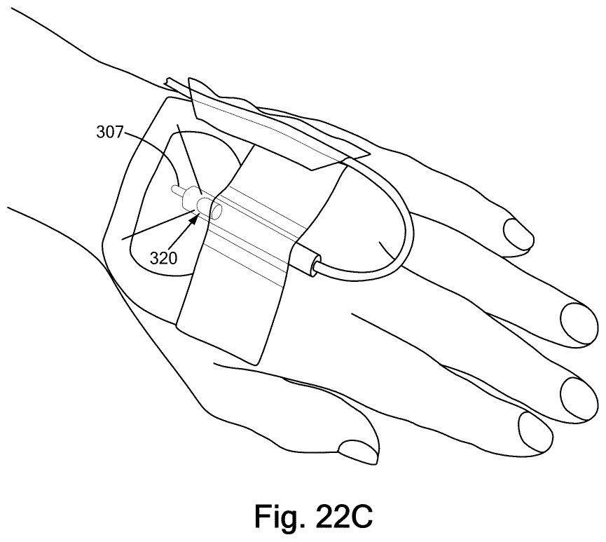

FIGS. 22A-22C show use cases of deployment on the patient skin surface 312. The illumination source 320 may be disposed in any suitable orientation on top of or adjacent the needle 307 or catheter, for ensuring that the medication tube 348 or vessel and point of insertion are illuminated. Multiple tubes 348 may be employed.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An antimicrobial light-emitting device, comprising:
a skin-safe antimicrobial light source;
a power source; and
an attachment for use with catheter dressings.

2. The device of claim 1 wherein the light source emits light that is both antimicrobial and noncarcinogenic to the skin.

3. The device of claim 1 wherein the light source is comprised of at least one light-emitting diode (LED).

4. The device of claim 1 wherein the power source is connected to the light source.

5. The device of claim 1 wherein the attachment is configured for use with a catheter dressing.

6. The device of claim 1 wherein the attachment is configured for use with a catheter dressing to secure the light source.

7. The device of claim 1 wherein the attachment is configured to position the light source to bathe a region around a skin entry site of a catheter.

8. An antimicrobial light-emitting device, comprising:
a skin-safe antimicrobial light source;
a power source; and
an attachment for use with catheter securement devices.

9. The device of claim 8 wherein the light source emits light that is both antimicrobial and noncarcinogenic to the skin.

10. The device of claim 8 wherein the light source is comprised of at least one LED.

11. The device of claim 8 wherein the power source is connected to the light source.

12. The device of claim 8 wherein the attachment is configured for use with a catheter securement device.

13. The device of claim 8 wherein the attachment is configured for use with a catheter securement device to secure the light source.

14. The device of claim 8 wherein the attachment is configured to position the light to bathe the region around the skin entry site of a catheter.

15. A method of reducing catheter-related infections, comprising:
providing a skin-safe antimicrobial light-emitting device;
positioning the antimicrobial light to bathe the region around a skin entry site of a catheter; and
reducing the bacterial burden in the region around the skin entry site of a catheter based on an irradiation from the antimicrobial light.

16. The method of claim 15 wherein the antimicrobial light-emitting device further comprises a skin-safe antimicrobial light source, a power source, and an attachment for use with catheter dressings or catheter securement devices.

17. The method of claim 15 wherein the antimicrobial light kills bacteria in the region around the skin entry site of a catheter.
